(19) United States
(12) Patent Application Publication
(10) Pub. No.: US 2008/0146894 A1
(43) Pub. Date: Jun. 19, 2008

(54) SIGNAL SENSING IN AN IMPLANTED APPARATUS WITH AN INTERNAL REFERENCE

(76) Inventors: Cherik Bulkes, Sussex, WI (US); Stephen Denker, Mequon, WI (US)

Correspondence Address: QUARLES & BRADY LLP, 411 E. WISCONSIN AVENUE, SUITE 2040, MILWAUKEE, WI, 53202-4497, US

(21) Appl. No.: 11/959,952

(22) Filed: Dec. 19, 2007

Related U.S. Application Data
(60) Provisional application No. 60/870,649, filed on Dec. 19, 2006.

(51) Int. Cl. A61B 5/04

(52) U.S. Cl. 600/300

(57) ABSTRACT

An apparatus for sensing biological signals from an animal. The implanted apparatus includes at least one implantable electrode pair that is connected to a twisted pair of insulated conductors. The electrode pair is disposed at a first set of locations to sense biological signals. The twisted pair of insulated conductors is connected to an instrumentation amplifier via a passive network of filters. The amplifier amplifies the filtered biological signal from the electrode pair to provide an amplified differential signal. The amplifier has an internal voltage reference. Additionally, an energy source powers the apparatus without being connected to mains or an isolation transformer of medical equipment. A signal analysis module analyzes amplified differential signals to obtain at least one physiological parameter. The apparatus may also comprise a signal presentation module to display amplified signals and physiological parameters associated with the signal.

### CROSS REFERENCE TO RELATED APPLICATION

This application claims the benefit of U.S. Provisional Patent Application No. 60/870,649 filed on Dec. 19, 2006, the disclosure of which is hereby incorporated by reference as if set forth in its entirety herein.

### STATEMENT REGARDING FEDERALLY SPONSORED RESEARCH OR DEVELOPMENT

Not Applicable.

### BACKGROUND OF THE INVENTION

1. Field of Invention

The present invention relates to medical devices, which provide a means to sense physiological signals from nerves and muscles in humans. Specifically, the current invention relates to sensing physiological signals in their pristine form while avoiding irrelevant noise sources.

2. Description of the Related Art

In an exemplary case of electrical sensing and amplifying of physiological signals, the amplifier has competing electromagnetic signal sources that may cause deterioration of signal quality performance. Established methods use common mode rejecting amplifier designs, which reference the leads of a signal pair to a reference and a real or virtual ground. When the signals have amplitudes in the range of a few tens of mV, the performance of such solutions is good, as the operating voltage range is many orders of magnitude greater than the supplied signal. On the other hand, for biological signals encountered in an implanted system, the traditional techniques with an external ground are not optimal as the relatively smaller magnitude of the biological signals can be easily overwhelmed by noise.

In a conventional data acquisition system, the input bandwidth must be limited to avoid aliasing. Aliasing is the result of not having sufficient data samples available to distinguish a component with frequency content F from one with n×2 F. However, aliasing would become an issue only if sufficient energy is contained in higher frequencies. According to Shannon/Nyquist theorem, the sample frequency must be at least twice the lowest frequency component contained in the signal at the lowest amplitude of the dynamic range of the system.

The frequency range for ECG signals has traditionally included the line frequencies, 50 Hz and 60 Hz. In a traditional system, with an input pair and a common ground in an office, home or industrial environment, there is likely considerable line frequency content in the input signal, at the input amplifier and or sampling location. One solution would include a notch filter for 50 Hz and 60 Hz, or one broad band enough to filter out the band from 45-65 Hz. The notch filtering will introduce a non-linear effect from at least 22.5 Hz to 130 Hz resulting in system sensitivity reduction. Even a high Q filter will not avoid this issue. The other common line frequency for aviation and marine equipment is 400 Hz. However, this is generally high enough not to affect ECG signals. If there is no meaningful information contained in the filtered out band, there will not be any adverse issues with the filtering approach. In practical applications, that is almost never the case. Since important information is contained in those frequency bands, there is a need for a technique that includes the entire band from 10 Hz to 200 Hz so pristine biological signals can be acquired.

A further problem afflicting present-day devices relate to the rejection of amplitude modulated or burst electromagnetic fields. One source of burst line frequency noise is faulty, or poorly designed, appliances where the patient is in the close proximity of or in contact with a line frequency AC powered device. The patient actually is part of a direct or induced electrical pathway to ground. In contrast, to sense detection in the presence of continuous additive line frequency interference, the operation of the sensing circuit during amplitude modulated or burst electromagnetic interference (EMI) is probably more important to patient safety. Burst line frequency noise is a potentially dangerous situation for pacemaker-dependent patients because burst noise may inhibit stimulus generation in a cardiac control device. The potential hazard of continuous line frequency noise, in comparison to burst noise, is less precarious because continuous line noise will cause the device to pace asynchronously with respect to a spontaneous cardiac rate, but the device will still support the patient.

Yet another problem with prior art techniques is the usage of digital or active analog filtering in the front end circuit that is directly connected to electrodes. This exposes the internal circuits to the full noise amplitude and has the risk of running out of “dynamic range.” For example, if the amplifier output hits the rails (ground or supply), it is no longer linear, or amplifying. For example, given a normal signal range of 1-10 mV, gain=200, noise burst 100 mV and supply rail 5V, the output amplitude of the true signal is 200 mV to 1.0V and the noise signal in the output is 20V, which is well beyond the supply rail. The amplifier may simply peg at the rail, or oscillate between ground and 5V, without linear relation to the input signal.

In view of the foregoing discussion, there is a need for a system that can amplify biological signals from muscles and/or nerves without concomitantly amplifying the noise.

### SUMMARY OF THE INVENTION

In accordance with one aspect, an apparatus for sensing biological signals from an animal is provided. The apparatus can include at least one set of electrodes that is configured to be implanted in the animal and disposed at a first set of locations to sense biological signals from the first set of locations, a set of insulated conductors connected to the at least one set of electrodes, the set of insulated conductors formed in a configuration adapted to be substantially immune to electromagnetic interference, a network of filters connected to the set of insulated conductors, the network of filters configured to filter the sensed biological signals, an amplifier connected to the network of filters, the amplifier including an internal voltage reference and the amplifier configured to amplify the filtered biological signals to provide an amplified differential signal, an energy source powering at least the amplifier, the energy source configured to be substantially free of an externally grounded energy supply external to the animal, and a signal analysis module configured to receive the amplified differential signal and to analyze the amplified differential signal to determine at least one physiological parameter.

In accordance with another aspect of the invention, an implanted apparatus for sensing biological signals from an animal is provided. The apparatus can include at least one implanted electrode pair disposed at a first set of locations to sense biological signals. Each electrode pair can be connected to a pair of insulated conductors that are in turn connected to an instrumentation amplifier via a passive network of filters. The insulated conductors can be configured to avoid picking up of EMI noise. The amplifier can amplify the filtered biological signal from each of the electrode pairs to provide an amplified differential signal. The amplifier can have an internal voltage reference. Additionally, an energy source can power the apparatus without being connected to mains or an isolation transformer of medical equipment. A signal analysis module can analyze amplified differential signals to obtain at least one physiological parameter. The apparatus may also include a signal presentation module to display amplified signals and physiological parameters associated with the signal. The energy source may be a battery, an infrared source or a radio frequency source. The electrodes may be located inside a blood vessel, extravascular or transvascular and sense signals from various tissue locations.

Embodiments of the present invention can provide for the elimination of ground and associated noise sources by passive component and instrumentation amplifier design, elimination of DC and very low frequency noises by a high pass filter, elimination of common mode noise by a low pass filter over 500 Hz and a noise filter over 1 kHz. There can be no need for line frequency filtering with the elimination of traditional ground. Another aspect of the invention is the use passive filtering at the front end, before any active components are involved. Electronic circuitry can use light emitting diode (LED) or any other suitable diode as an internal reference with a constant 1.2 V or other suitable voltage. Additionally, the conductors connecting to the electrodes can be paired to avoid the formation of EMI pickup loops.

### BRIEF DESCRIPTION OF DRAWINGS

FIG. 1 is a schematic of a sensing amplifier system with a three-conductor path;

FIG. 2 is a schematic of a sensing amplifier system with a two-conductor path;

FIG. 3 is a schematic of a sensing amplifier with an internal reference and a high pass filter to reject DC and low frequencies;

FIG. 4 shows the frequency response of band pass filtering employed by an embodiment of the invention;

FIG. 5 is a schematic of a sensing amplifier with internal reference and signal pre-filters;

FIG. 6 is a schematic of an internal reference module; and

FIG. 7 is a block diagram of an implantable apparatus connected to a signal analysis module and a signal presentation module or a signal recording module.

### DETAILED DESCRIPTION

Although the present invention may be described in the context of cardiac pacing and of implanting a sensing circuit in a vein or artery of the heart, the present apparatus can be employed to sense signals from muscles and/or nerves in other areas of the human body. In addition to cardiac applications, the sensing apparatus can provide brain signal sensing, for treatment of Parkinson's disease or obsessive/compulsive disorder for example. The transvascular electrical sensing also may be applied to signals from muscles, the spine, the gastro/intestinal tract, the pancreas, and the sacral nerve. The apparatus may also be used for sensing in GERD treatment, endotracheal sensing, pelvic floor sensing, sensing for obstructive airway disorder and apnea, and molecular therapy delivery sensing.

An aspect of embodiments of the invention is the use of a plurality of electrode pairs disposed at a first set of location for the signal sensing. It should be understood that each electrode pair in close proximity or farther apart is included in the set. Further, if more electrode pairs are involved, the term set should encompass all such paired locations as well. In the subsequent description, signal sensing and amplification is described at only one electrode pair for the sake of convenience and it should be understood without loss in generality, that the present invention can be configured to sense from a plurality of locations.

A signal amplifier and associated electronics that do not require an external ground is described. The signal amplifier with an internal ground will only see common mode signals especially when the signal pair is either a coaxial or a twisted pair.

First, referring to the FIG. 1, a three-conductor system 10 is described. System 10 includes a pair of implantable electrodes 12, 14 connected on lines 16, 18 to an amplifier 20. Amplifier 20 is connected on line 22 to a positive power supply 24, is connected on line 26 to external ground 28, and has an output 30. In this case, Vₒᵤₜ 32 is the voltage of the output signal, Vₐ 34 and V_{b} 36 are the voltages sensed by electrode pair 12, 14, V_{signal} 38 is the voltage of signal 40, Vₙₒᵢₛₑ 42 is the voltage of noise 44, and Gain is the voltage gain of the instrumentation amplifier 20. Now, Vₒᵤₜ=Gain (Vₐ−V_{b}); where Vₐ=V_{signal}+Vₙₒᵢₛₑ and V_{b}−Vₙₒᵢₛₑ. In the difference mode, we can subtract out the Vₙₒᵢₛₑ, with “Gain (Vₙₒᵢₛₑ−Vₙₒᵢₛₑ)=0”, leaving Vₒᵤₜ=Gain (Vₐ−V_{b}).

### EMI Noise Mitigation:

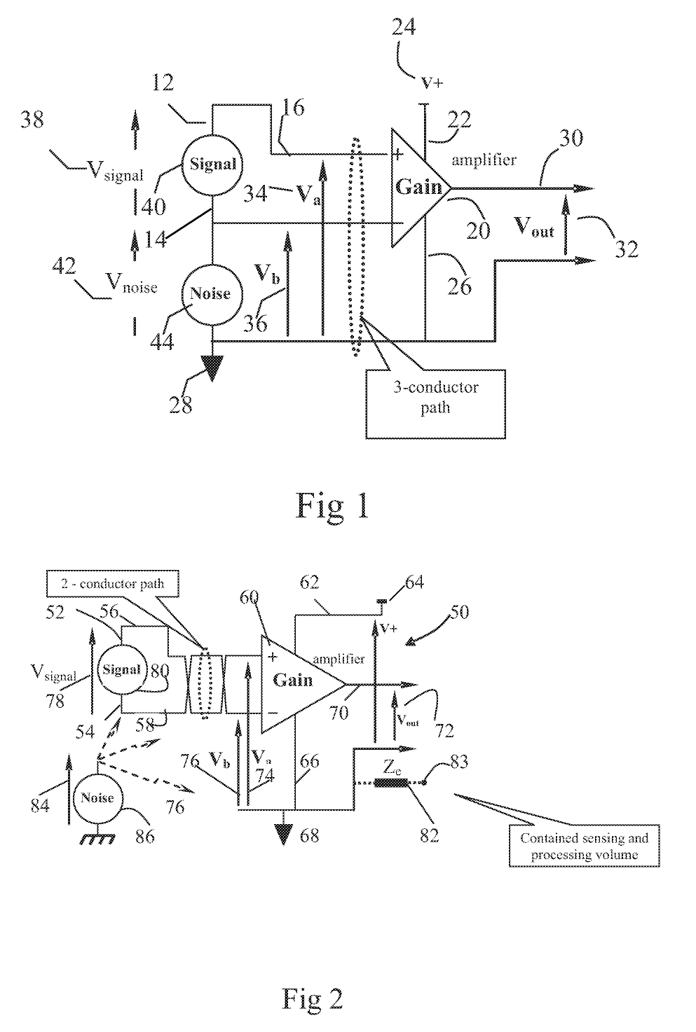

If the main signal leads, providing Vₐ and V_{b} are contained within a space or volume with noise sources external to that volume as would be the case in an implanted system, the reference or ground lead may be removed with a concomitant performance improvement of the system 50 shown in FIG. 2. System 50 includes a pair of implantable electrodes 52, 54 connected on lines 56, 58 to amplifier 60. Amplifier 60 is connected on line 62 to a positive power supply 64, is connected on line 66 to internal ground 68, and has an output 70. By removing the external reference or ground, the signal lines maybe exposed to common mode noise. However, without a path to reference this noise, a common mode circuit cannot be formed. This results in the original signals being presented to the amplifier. By arranging the two signal leads 56, 58 in a twisted pair fashion, it can be ensured that input conductor impedance for the signal amplifier 60 is equal for both the leads with equal noise exposure.

Vₒᵤₜ 72 is the voltage of the output signal, Vₐ 74 and V_{b} 76 are the voltages sensed by electrode pair 52, 54, V_{signal} 78 is the voltage of signal 80, and Gain is the voltage gain of the instrumentation amplifier 60. In this case, Vₒᵤₜ=Gₐᵢₙ (Vₐ−V_{b}), where Vₐ−V_{b}=V_{signal}; and Vₒᵤₜ=Gₐᵢₙ (V_{signal}). It should be noted that the twisted pair is a specific case of the more general helical configuration.

In the FIG. 2, Ze 82 is a virtual component between internal ground 68 and an enclosure 83 containing system 50. Ze 82 represents the impedance to the enclosing volume. When the enclosing volume has low impedance to the noise generator it will form an electrostatic shield, whose effectiveness increases proportionally to the conductivity of that environment.

There are other methods of mitigating electromagnetic interference. One of the methods include running wires in close proximity, for example, a mm or less, relative to the wavelength of the EMI field, e.g., 5000 km for a EMI field of 60 Hz, from which immunity is needed. Another method of mitigating the effects of EMI filed is a multifilar helical coil, where the insulated conductors run parallel, but at the same time form helical coils. This option offers additional combinations of tuning dimensions, such as conductor/helix diameter ratio, spacing, pitch, and spacing pattern. Note that the coil does not need to be evenly spaced.

Noise voltage 84 of noise 86 can still be injected within each individual conductor and present an unbalanced noise component to the amplifier 60 where it will be amplified and spoil the original signal. Depending on location and application, the contributions of unbalanced noise must be considered before choosing this method as described next.

### Groundless Signal Amplifier/Detector:

There are a few considerations in a practical implementation of the previous circuit. First, there are DC considerations. Second, there is an internal reference consideration. Third, there are filtering considerations. In the following, each of these is described in detail.

### DC Considerations:

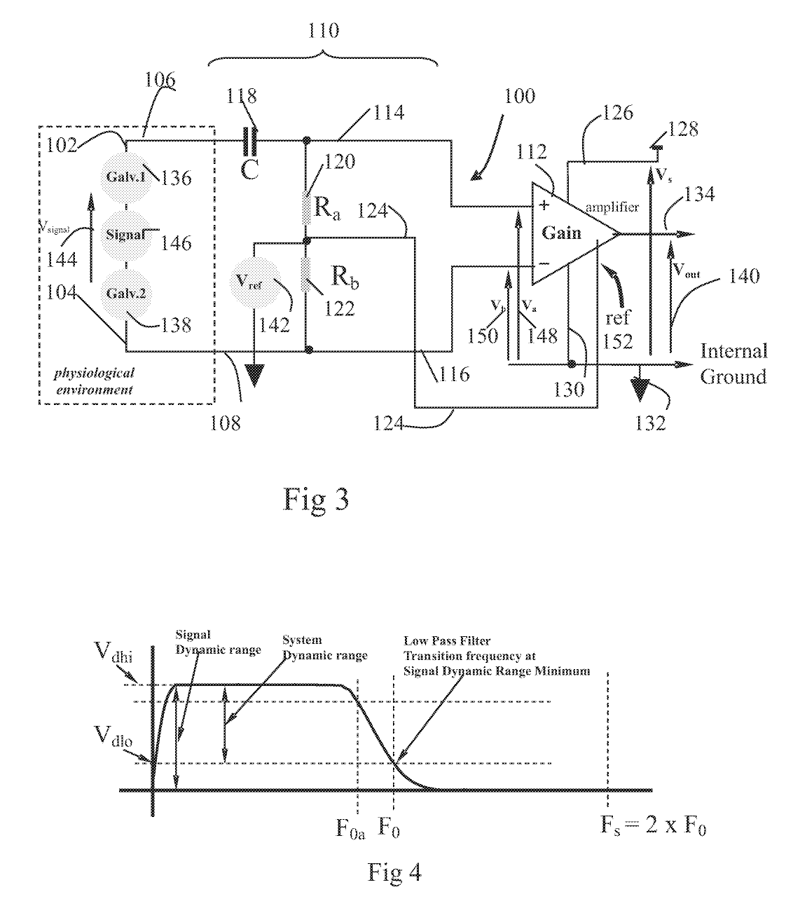

FIG. 3 illustrates components for the DC considerations. System 100 includes a pair of implantable electrodes 102, 104 connected on lines 106, 108 to filter network 110, which is connected to amplifier 112 by lines 114, 116. Line 116 is connected to line 108, which means that lines 116 and 108 can be considered a single line. Filter network 110 includes a capacitor 118 having a capacitance C, a resistor 120 having a resistance Ra, and a resistor 122 having a resistance Rb. Capacitor 118 is connected between lines 106 and 114. Resistor 120 is connected between lines 114 and a line 124; and resistor 122 is connected between line 124 and line 116. Amplifier is connected on line 126 to a positive power supply 128, is connected on line 130 to internal ground 132, and has an output 134.

At the interface between electrode and tissue, a galvanic system is formed with a DC potential. If there is complete symmetry in this circuit from electrode-1 to electrode-2, then the sum of all the contact potentials will cancel. However, if the materials used are dissimilar, the electrode/tissue and or the electrode/blood interface will yield potentially different galvanic generators that do not cancel. In this case, the input amplifier is presented with the source voltage of interest along with the galvanic voltage difference. This galvanic component is relatively static, but it could potentially be modulated with body or organ movement, as the electrode may wander between touching the vessel wall and the blood pool thereby presenting a varying “DC” voltage. The variance over time is expected to be synchronous with the movement, and thus in the sub 2 Hz range, if respiratory and cardiac movements are included. Another DC issue stems from the amplifier itself, which will require a DC current bias into or out of the input terminals. In MOSFET amplifiers, this “bias current” is very small, but doubles with every ten degree Celsius (10° C.)in temperature rise. Also, this current can have an offset, leaving a differential current that can spoil the balance of a high impedance circuit. This problem can be substantially alleviated by providing a form of AC coupling with the electrodes, and a DC current path for the bias currents.

The AC coupling performs two functions. The first function is DC decoupling from the galvanic voltages, Galv.1 136 and Galv.2 138, and the second function is to form a high pass filter with a corner frequency of F_{HP}=½π RC, where R=Ra+Rb.

The bias and offset currents are in the order of 10⁻⁹ to 10⁻⁸ A, and with path resistances of e.g. 100 kOhm, still yield 0.1 to 1.0 mV. Since source voltages are in order of 0.5-10 mV, these bias and offset voltages are not negligible. Therefore, in this embodiment, the amplifier specification selection should be such that these currents are low enough to allow for reasonably high input resistance values in the order of 100 kOhm or better for Ra and Rb (FIG. 3).

Careful selection of Ra and Rb will yield an acceptable low bias current offset voltage component (V_{offset}=I_{offset}×Ra, where Ra=Rb), and a proper F_{HP} (high pass filter frequency). The traditional corner frequency range for F_{HP} is in the order of 0.5 Hz to 2.0 Hz, but other values can be selected depending on spectral regions of interest.

A natural feature that helps our proposed implementation is the relatively low impedance of the tissues involved, typically 300 to 120 Ohm between, for example, 5 mm spaced electrodes. Thus, in order to create a net 1 mV across such an impedance, energy density of 0.4 mW/m would be needed with the energy contained from 0-1 kHz.

### Reference Considerations

In order to incorporate a floating AC coupled signal, such as the one shown in FIG. 3, it is desirable to provide a reference point. If the signal is expected to be symmetrical, a V_{ref}=Vs/2 can be selected, thus allowing Vₒᵤₜ to swing between ground and Vₛ, with a rest point at V_{ref}, where Vₒᵤₜ is the output voltage 140, V_{ref} is the reference voltage 142, and Vs is the signal voltage 144 of signal 146. This reference input is provided to the output stage of the amplifier 112. Commercially available instrumentation amplifiers do have a provision to receive reference input for the amplifier output stage. The original input signal can now be presented at the output as: Vₒᵤₜ=V_{signal}×Gain×F, where F is a high pass filter function.

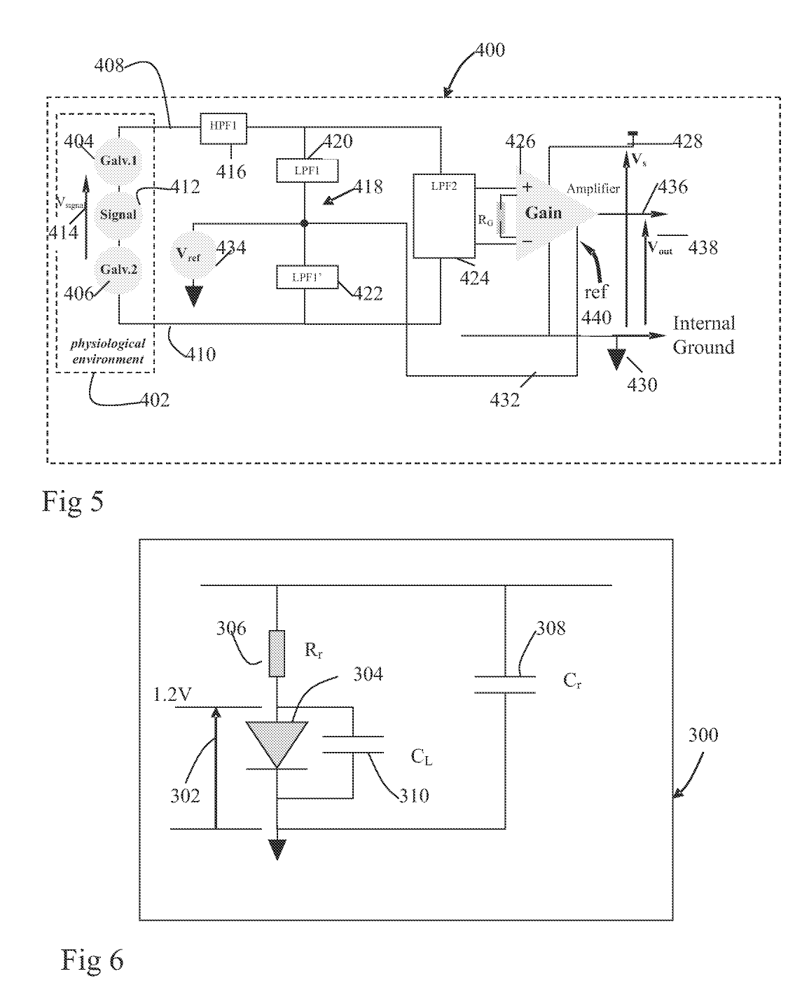

Additional details for the internal reference are provided in FIGS. 5 and 6. Referring now to FIG. 6, an internal reference module 300 produces a reference voltage 302 of 1.2 volts (V) by using a light emitting diode (LED) 304 that has a series resistor 306 having a resistance Rᵣ and two parallel capacitors 308, 310 having capacitances Cᵣ and C_{L}, respectively. Two factors help in an LED being used as a stable reference voltage. First, the electronics module containing signal amplifier/detector as a part is in an intravascular environment, wherein the blood pool provides a seal to control the ambient environment. Second, the thermal properties of this environment are relatively constant at the internal body temperature. Therefore, it can be shown from the fundamental considerations that the voltage drop across the LED would remain sufficiently constant at relatively constant temperature.

### Filtering Considerations

If there is no meaningful information contained in the filtered out band, there will not be any adverse issues with the filtering approach. In practical applications, that is, however, rarely the case. Since important information is contained in those frequency bands, an embodiment is tailored to include the entire band from 10 Hz to 250 Hz. For robustness reasons even a wider range of frequencies (e.g., 2 Hz-500 Hz) can be used. With this consideration, the fast rise time of the sinus node signals containing high frequency content in the 100-250 Hz range can be easily accommodated in their pristine form. Additionally, by including these frequency components, the natural physiological signals can be easily distinguished from background signals, such as noise, voluntary and involuntary muscle movement, etc.

FIG. 4 shows the frequency response of the band pass filtering used in embodiments of the invention. The voltages VDhi and VDlo are used represent the high and the low voltages determining the system dynamic range. The system dynamic range always excludes inherent system noise where as the signal dynamic range is higher since it also includes noise components. The VDlo is the voltage at which the signal falls below the intrinsic noise floor of a conventional system. FOA is the lowpass cut-off frequency and FO is the frequency at which the filter output goes below VDlo. In theory, the sampling frequency (FS) must be at least 2× the lowest frequency component contained in the signal at the lowest amplitude of the system dynamic range. This means that the filter transition band (F0a to F0) must be included when determining this lowest frequency. The high frequency filtering helps in AC coupling while the low frequency filtering helps in minimizing the noise components in the signal.

FIG. 5 illustrates a system 400 according to an embodiment of the invention. A physiological environment 402 is shown to contain the galvanic voltage one 404 (Galv.1) and galvanic voltage two 406 (Galv.2) formed at the tissue electrode intersections of two electrodes 408, 410. The biological signal source that would be sensed is shown as the signal generator 412 with an associated signal voltage 414 V_{signal}. The source may also have associated source impedance (Z_{source}), which is not shown.

Between the biological environment and the signal amplifier, a network of filters, which for example can comprise at least three filters, is provided to perform various functions. The first of these filters is a high pass filter 416 to substantially block DC and low frequencies up to a prespecified cut-off (e.g., 2 Hz). This high pass filter 416 comprises of passive elements with capacitance and resistance, where resistance may be obtained by a combination of resistors, and source impedance in series. A suitable low pass filter 418 (LPF1) is configured to suppress common mode noise. Low pass filter 418 comprises of passive elements 420, which can comprise capacitance C and resistance R, and their symmetrical counter parts 422 (LPF1′). A second low pass filter 424 (LPF2) is configured to reject high frequency noise signals. Low pass filter 424 filter cam comprise passive elements capacitor and resistors in series. EM broadband ambient noise from appliances and other equipment could swamp the input circuit and consume dynamic range. This needs to be filtered out. A low pass filter LPF2 with a cut-off at 1 kHz frequency can be selected since the EM noise is broad band, but its energy is rather low below 10 kHz and can be effectively filtered out.

System 400 further includes an amplifier 426 connected to the network of filters (i.e., filters 416, 418, and 424). Amplifier 426 is connected to a positive power supply 428, is connected to an internal ground 430, and receives an internal reference 432 provided by an internal reference module 434. Amplifier 426 has an output 436 having an output voltage 438.

### Other Considerations:

For ECG signals obtained by direct connection to the cardiac venous vessel wall or muscle tissue, the signal path between the two or more input electrodes should exclude any electromagnetic pickup loop, for example, by twisting the lead and or wire pairs. Therefore, symmetrical layouts are favored.

Absence of a traditional ground is a significant departure from the prior art and it has obviated the need for notch filtering and other kinds of signal degrading processes. Another aspect of the invention as already mentioned is the use passive filtering at the front end, before any active components are involved. As a result, physiological signals devoid of the traditional noise are obtained.

### Integrated System:

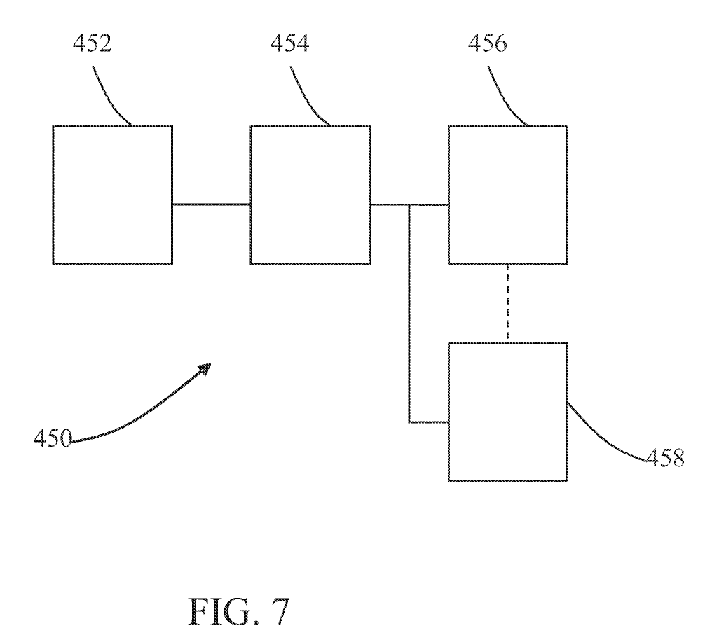

Referring now to FIG. 7, a system 450 comprises a sensing, filtering, and amplification module 452 connected to a signal analysis module 454, which can be connected to a recording module 456 and/or a signal presentation module 458.

Module 452 can be comprised of system 100 of FIG. 3, system 400 of FIG. 5, or other like system including at least one implanted electrode pair disposed at a first set of locations to sense biological signals. Each electrode pair can be connected to a twisted pair of insulated conductors that are in turn connected to an instrumentation amplifier via a passive network of filters. The amplifier amplifies the filtered biological signal from each of the electrode pairs to provide an amplified differential signal from the first set of locations. The amplifier has an internal voltage reference. Additionally, an energy source powers the apparatus without being connected to mains or an isolation transformer of medical equipment. Module 452 senses a biological signal, filters the sensed biological signal, and amplifies the filtered biological signal. The output of module 452 is an amplified differential signal.

Signal analysis module 454 receives the amplified differential signal from module 452. Signal analysis module 454 is configured to analyze the amplified differential signal to determine at least one physiological parameter of the biological signal sensed by module 452. For example, in a cardiac application of the apparatus, the parameter obtained may be heart rate. In general, the parameter extracted from the analysis module may be used to provide a therapy (e.g., stimulation) to a patient. The signal analysis module may include a hysteresis based signal transition detector or a zero-crossing detector. Additional signal processing algorithms for detection and identification of biological signals may be used as part of the signal analysis module.

The recording module 456 can be connected to signal analysis module 454 to record the amplified differential signal and/or the at least one physiological parameter. The recording module 456 can be configured to communicate (e.g., wirelessly via remote telemetry) to present saved and/or live data to a further module (not shown).

Additionally, presentation module 458 can be configured to receive the amplified differential signal and/or the at least one physiological parameter. Presentation module can display amplified signals and physiological parameters associated with the sensed biological signal. The display module may be accessed remotely via telemetry at a readout station (e.g. doctor's office). Alternatively, the signal presentation module may provide a print out of a recording of the signal. In other alternatives, recorded signal may be stored in an electronic form for a later retrieval. The presentation module can be located outside the body and be configured to wirelessly communicate with signal analysis module 454 and/or module 452. Moreover, the presentation module 458 can be configured to wirelessly communicate with the recording module 456, which can be configured to transmit saved data to the presentation module 458.

An energy source for any of modules 452, 454, 456, and 458 can be a battery, an infrared source or a radio frequency source. The energy source is not connected to the mains or via an isolation transformer of medical equipment. This is to avoid connecting to any external grounding as mentioned earlier to avoid introduction of noise.

In summary, embodiments include leads that minimize EMI noise, passive filtering prior to signal amplification with relatively high frequency high pass filter with a cut-off frequency in the range of 20 to 70 Hz combined with a relatively high low pass filter with the cut-off frequency above 300 Hz, an amplifier with an internal voltage reference, and avoidance of connecting the energy source to an external ground.

In one embodiment, at least a pair of electrodes may be located inside a blood vessel and sense signals from various tissue locations. In another embodiment, such as in the case of most nerve stimulators, the electrode location may be extravascular. In another embodiment, the electrode pair is implanted under the skin for detecting biological rhythms. In another embodiment, the electrode pair may be part of sensing pressure in the heart. In general, the invention is applicable to signal amplification beyond nerve and cardiac applications where physical parameters are converted to electrical signals and could be affected by noise. Thus, the invention is applicable for sensing applications of all physiologic data including intravascular, extravascular and transvascular. The applicability of the invention further includes systems which are temporary and are both in the body and extend out of the body, such as temporary pacing leads.

The foregoing description was primarily directed to preferred embodiments of the invention. Although some attention was given to alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

We claim:

1. An apparatus for sensing biological signals from an animal, the apparatus comprising:
at least one set of electrodes that is configured to be implanted in the animal and disposed at a first set of locations to sense biological signals from the first set of locations;
a set of insulated conductors connected to the at least one set of electrodes, the set of insulated conductors formed in a configuration adapted to be substantially immune to electromagnetic interference,
a network of filters connected to the set of insulated conductors, the network of filters configured to filter the sensed biological signals;
an amplifier connected to the network of filters, the amplifier including an internal voltage reference and the amplifier configured to amplify the filtered biological signals to provide an amplified differential signal;
an energy source powering at least the amplifier, the energy source configured to be substantially free of an externally grounded energy supply external to the animal; and
a signal analysis module configured to receive the amplified differential signal and to analyze the amplified differential signal to determine at least one physiological parameter.

2. The apparatus of claim 1 further comprising at least one of a signal presentation module configured to display the amplified differential signal and a recording module to at least one of present and record the amplified differential signal.

3. The apparatus of claim 1, wherein the energy source is one of a battery, an infrared source, and a radiofrequency source.

4. The apparatus of claim 1, wherein the location of the at least one set of electrodes is one of intravascular, extravascular, and transvascular.

5. The apparatus of claim 1, wherein the at least one physiological parameter is a heart rate of the animal.

6. The apparatus of claim 1, wherein the internal reference is provided by a diode configured to produce a substantially constant voltage.

7. The apparatus of claim 6, wherein the diode is a light emitting diode.

8. The apparatus of claim 6, wherein the internal reference is substantially 1.2V.

9. The apparatus of claim 1, wherein the network of filters comprises at least one high pass filter and at least one low pass filter.

10. The apparatus of claim 9, wherein the at least one high pass filter is a single order filter.

11. The apparatus of claim 9, wherein each of the at least one high pass filter and the at least one low pass filter is a passive filter comprising at least one capacitor and at least one resistor.

12. The apparatus of claim 1, wherein the set of insulated conductors comprises a helical pair of insulated conductors.

13. The apparatus of claim 1, wherein the set of insulated conductors run in parallel and in close proximity of under 3 mm.

14. The apparatus of claim 1, wherein the set of insulated conductors comprises insulated conductors that are in a multifilar form, run in parallel, and have a helical formation.

15. The apparatus of claim 14, wherein the multifilar form is tuned by changing dimensions of at least one of conductor-to-helix diameter ratio, spacing, pitch, and spacing pattern.

16. The apparatus of claim 1, wherein the signal analysis module comprises at least one of a hysteresis based signal transition detector and a zero-crossing detector.

17. An apparatus for sensing biological signals from an animal, the apparatus comprising:
at least one implantable electrode pair disposed at intravascular locations to sense biological signals;
a twisted pair of insulated conductors connected to the at least one implantable electrode pair;
a passive network of filters connected to the twisted pair of insulated conductors, the passive network of filters configured to filter the sensed biological signals;
an instrumentation amplifier connected to the passive network of filters, the instrumentation amplifier configured to amplify the filtered biological signals to provide an amplified signal, wherein the amplifier has an internal voltage reference;
an energy source powering the apparatus without being connected to mains or an isolation transformer of medical equipment; and
a signal analysis module configured to analyze the amplified signal.

18. The apparatus of claim 17, wherein the internal voltage reference is generated by a diode.

19. The apparatus of claim 17, wherein the passive network of filters comprises a low pass filter, a high pass filter, and a noise filter.

20. An apparatus for sensing biological signals from an animal, the apparatus comprising:
at least one implantable electrode pair disposed at a first set of locations to sense biological signals;
a pair of insulated conductors connected to the at least one implanted electrode pair, the pair of insulated conductors configured to minimize sensitivity to electromagnetic interference;
a filter network connected to the pair of insulated conductors and configured to provide a filtered biological signal, the filter network comprising at least one high pass filter and at least one low pass filter, the high pass filter configured to provide AC coupling and the low pass filter configured to provide noise rejection;
an internal reference module configured to provide an internal voltage reference;
an instrumentation amplifier connected to the filter network, the instrumentation amplifier configured to receive the internal voltage reference and to amplify the filtered biological signal to provide an amplified biological signal;
an energy source powering the apparatus, the energy source configured to be substantially free of a wired connection to an energy supply external to the animal;
a signal analysis module configured to analyze the amplified differential signal to extract at least one physiological parameter;
wherein the implantable apparatus is substantially free of a wired ground connection to a ground external to the implantable apparatus.
